(19) United States
(12) Patent Application Publication
(10) Pub. No.: US 2020/0121050 A1
(43) Pub. Date: Apr. 23, 2020

(54) VIRTUAL REALITY KIT

(71) Applicant: LIMBIX HEALTH, INC., Palo Alto, CA (US)

(72) Inventors: Benjamin David Lewis, Los Altos, CA (US); Scott Benjamin Satkin, Palo Alto, CA (US); Jonathan Warren Sockell, Palo Alto, CA (US); John Luke Chuter, Portola Valley, CA (US); Benton Gabriel Chuter, La Jolla, CA (US)

(21) Appl. No.: 16/658,013

(22) Filed: Oct. 18, 2019

Related U.S. Application Data
(60) Provisional application No. 62/748,294, filed on Oct. 19, 2018.
(60) Provisional application No. 62/805,851, filed on Feb. 14, 2019.

(51) Int. Cl. A45C 13/02; A45C 11/00; A45C 15/00; H02J 7/00; A61L 2/10; A61L 2/26

(52) U.S. Cl. CPC A45C 13/02; A45C 11/00; A45C 15/00; H02J 7/0042; A61L 2202/11; A61L 2/26; A45C 2011/003; A45C 2013/025; A61L 2/10

(57) ABSTRACT

A virtual reality (VR) kit for storing and transporting components of a VR system is described. The VR kit includes receptacles for receiving and/or retaining positions of one or more of a VR headset, a tablet device, a controller, and a charging module. The VR kit can also include receptacles for receiving and/or retaining positions of a set of headphones, a set of earphones, and other material associated with use of a VR system. The VR kit includes a handle that maintains balance of the system during transportation the components of a VR system, while allowing a user to carry other objects. The VR kit is also designed for use in a clinical setting, by including materials that promote sanitization of components of a VR system. The VR kit is compact to facilitate storage of multiple units of the system in small spaces.

### CROSS-REFERENCE TO RELATED APPLICATIONS

The present application claims the benefit of the U.S. Provisional Patent Application No. 62/748,294 filed on Oct. 19, 2018, and U.S. Provisional Patent Application No. 62/805,851 filed on Feb. 14, 2019, which are all hereby incorporated by reference in their entirety.

### BACKGROUND

The cost of mental health conditions is $1 trillion in the U.S., with $150 billion spent annually on treatment of mental health conditions. Even though one in five Americans experiences symptoms of a mental health condition each year, treatment of mental health conditions is still insufficient in many ways. Typically, there are over six-month long waitlists to receive treatment, treatment options are limited, and therapists are often overworked, which reduces quality of treatment. Use of digital content has been found to be a viable treatment option in relieving therapist burden; however, content quality is limited by insufficiencies in methods and systems to generate high quality content, with respect to output device limitations and/or usage of such devices in a clinical environment.

The present application claims the benefit of the U.S. Provisional Patent Application No.

Furthermore, in terms of hardware involved in providing digital content to patients, current solutions lack functionality for facilitating use of such hardware in clinical environments. For instance, current systems lack features for improving portability, storage versatility, sanitization, and transition between in-use and storage modes within clinical environments. There is no system solution that facilitates treatment of mental health conditions with digital content, while addressing the issues described above.

### SUMMARY

Transportation, use, and storage of components of a virtual reality system designed for treatment of mental health conditions can present challenges, in relation to portability, storage versatility, sanitization, charging, and transitioning between different modes of use.

Embodiments of a virtual reality (VR) kit for storing and transporting components of a virtual reality system are shown and described. The VR kit includes receptacles for receiving and/or retaining positions of one or more of a VR headset, a tablet device, a controller, and a charging module. The VR kit can also include receptacles for receiving and/or retaining positions of audio output devices (e.g., a set of headphones, a set of earphones, etc.), and other components associated with use of the VR system, particularly for components relevant to use of a VR system in a clinical environment. The VR kit includes a handle that maintains balance of the VR kit during transportation of components of a VR system, while allowing a user to carry other objects. In one embodiment, the handle is aligned with the center of gravity of the VR kit during one or more modes of operation. In one embodiment, the VR kit can include an electrical component that interacts with a component of a VR system (e.g., in order to provide charging functionality). The VR kit is also designed for use in a clinical setting by including materials that promote sanitization of components of a VR system. The VR kit is compact to facilitate storage of multiple units of the system in small spaces.

### BRIEF DESCRIPTION OF THE DRAWINGS

FIG. 1A is an isometric view of a virtual reality kit, in accordance with one or more embodiments.

FIG. 1B is a front view of a virtual reality kit, in accordance with the embodiment of FIG. 1A.

FIG. 1C is a side view of a virtual reality kit, in accordance with the embodiment of FIG. 1A.

FIG. 1D is an isometric view from the top right of a VR kit, according to one or more embodiments.

FIG. 2 illustrates a first (top left), second (top right), third (bottom left), and fourth (bottom right) embodiment of a base of a VR kit, according to one or more embodiments.

FIG. 3 illustrates a first (top left), second (top right), and third (bottom middle) embodiment of a VR kit, according to one or more embodiments.

FIG. 4 illustrates a first (top left), second (top right), third (bottom left), and fourth (bottom right) embodiment of a handle of a VR kit, according to one or more embodiments.

FIG. 5A illustrates a first handle position (top) and a second handle position (bottom) of a first embodiment of a VR kit, according to one or more embodiments.

FIG. 5B illustrates a first handle position (top) and second handle position (bottom) of a second embodiment of a VR kit, according to one or more embodiments.

FIG. 6 is an isometric view of a virtual reality kit including a case, in accordance with one or more embodiments.

FIG. 7A illustrates a schematic of an embodiment of a virtual reality kit including a shield for protecting lenses of a head mounted display, where the shield is optionally transitionable between different configurations, according to one or more embodiments.

FIG. 7B illustrates a schematic of another embodiment of a virtual reality kit including a shield for protecting lenses of a head mounted display, where the shield is optionally transitionable between different configurations, according to one or more embodiments.

FIG. 8 illustrates a schematic of a virtual reality kit including a tablet positioning mechanism coupled to a handle that transitions the tablet positioning mechanism between different configurations, according to one or more embodiments.

FIG. 9 illustrates an isometric view of the VR kit 900, according to one or more embodiments.

FIG. 10A illustrates an isometric view of a VR kit 1000, according to one or more embodiments.

FIG. 10B illustrates a front view of the VR kit 1000, according to one or more embodiments

FIG. 10C illustrates an isometric view of the VR kit 1000 with a foam layer 1090, according to one or more embodiments.

FIG. 10D illustrates an isometric view of the VR kit 1000 with an obstructed view of electric components integrated into the VR kit 1000, according to one or more embodiments.

FIG. 11A illustrates an isometric view of a VR kit 1100 with a vertically oriented receptacle, according to one or more embodiments.

FIG. 11B illustrates an isometric view of a VR kit 1100 with an alternate handle 1130, according to one or more embodiments.

FIGS. 12A-B illustrates views of a VR kit 1200 with a handle extending upward, according to one or more embodiments.

FIGS. 12C-D illustrates views of a VR kit 1200 with a removable top 1250, according to one or more embodiments.

The figures depict various embodiments for purposes of illustration only. One skilled in the art will readily recognize from the following discussion that alternative embodiments of the structures and methods illustrated herein may be employed without departing from the principles described herein.

### DETAILED DESCRIPTION

### 1. Overview

A virtual reality (VR) kit is configured to store and transport components of a virtual reality system. As described in more detail below, in embodiments, a VR system associated with the VR kit can include a headset, a tablet device, and a controller. A VR system can include additional components such as a charging cable, a set of headphones, a sanitization system, etc.

Embodiments of the VR system may be used in a clinical setting or other institutional setting, such as a hospital, for patient diagnosis, treatment, therapy, and/or education. In more detail, it may be difficult for a patient to access a VR system in a clinical setting because many patients have difficulty walking, or may not be able to effectively receive content provided by the VR system without guided provision of such content. Additionally or alternatively, it can be burdensome (e.g., physically, from a time perspective, from a financial cost perspective) and/or dangerous to patient health to transport a patient across a clinical environment to a location where a VR system is stored. As such, it may be beneficial for clinical staff, such as nurses or other healthcare providers to bring a VR system to a patient.

However, because VR systems typically have multiple disparate components, they can be difficult to transport, especially when an individual is carrying other items, such as medical charts, IV stations, patient medicine, etc. The components of a VR system are also delicate and expensive, and they should be stored and transported with caution.

Furthermore, with respect to clinical use or other shared-use settings, components of a VR system may be used by multiple patients throughout the course of a day. As such, the VR system should be sanitized (or easily sanitizable) between use sessions. VR systems may be sanitized using alcohol wipes, sprays, and/or other disinfecting agents. Sanitization can thus be inconvenient and time consuming, especially if a VR storage system is not compatible with the sanitization method. The difficulties of storing, transporting, and sanitizing a VR system can deter hospitals from using VR systems as a medical diagnosis and treatment tool, negatively affecting patients. Embodiments of the systems described also provide solutions that facilitate sanitization of VR kit components in an efficient manner.

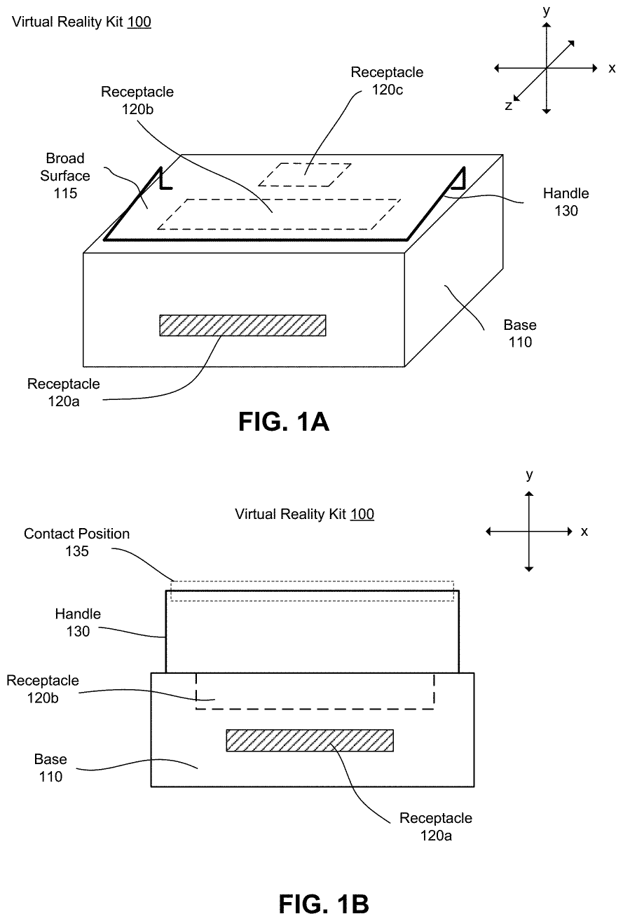

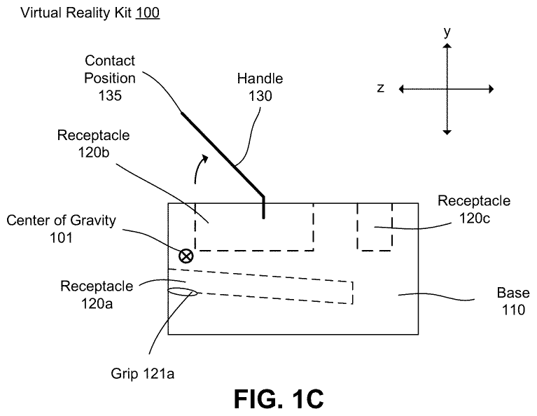

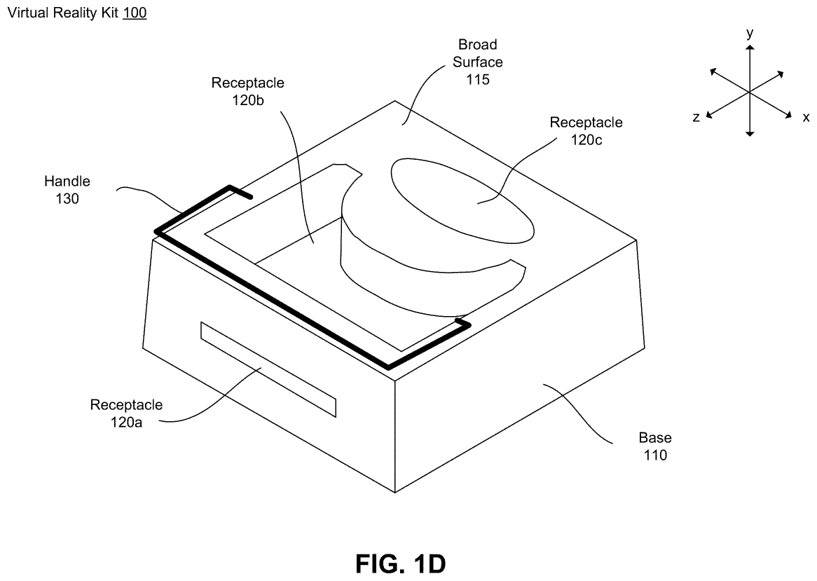

FIGS. 1A-1D illustrate various views of a virtual reality kit 100, in accordance with one or more embodiments. The virtual reality kit 100 shown in FIGS. 1A-1D includes a base 110 with receptacles (e.g., 120a, 120b, 120c) and a handle 130. In other embodiments, the VR kit 100 includes fewer or greater components than described herein. The components can also function in a different manner than described below.

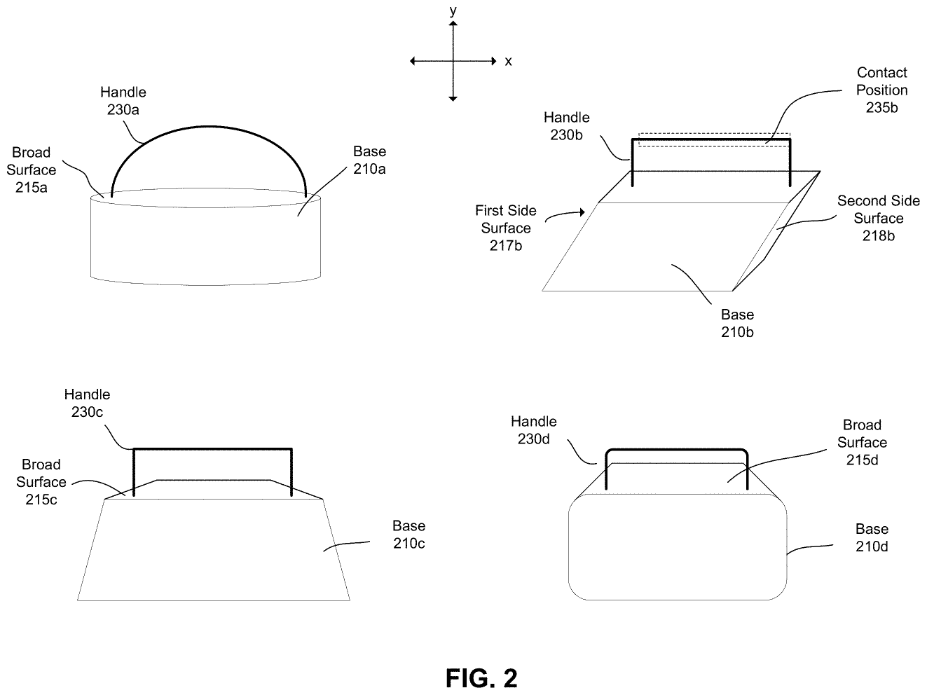

FIG. 1A is an isometric view of the virtual reality kit 100, according to one or more embodiments. The base 110 functions to transport and/or store a virtual reality system which can include a VR headset, a tablet device, and a controller. Shown in FIG. 1A, the base 110 is primarily rectangular prismatic (or approximately rectangular prismatic) in form factor, for transportation and storage purposes. The base 110 can be conveniently stacked with other items or other virtual reality kits 100 to minimize the space occupied by one or more virtual reality kits 100. In alternative embodiments, the base 110 can have another predominating form factor suited to transporting and storing a virtual reality system, described below in relation to FIG. 2.

The base 110 includes one or more receptacles for retaining components of a virtual reality system. A receptacle 120a is located on the front of the base 110, where the front of the base 110 is shown in a plane parallel to the x-y plane in FIG. 1A. The receptacle 120a is configured to receive a tablet device. In alternative embodiments, the receptacle 120a may house another electronic device, such as a cell phone, used to communicate with a VR headset and/or store information related to a patient and/or a VR system. The receptacle 120a can be recessed, at least partially, into the base 110 along the z-axis. The receptacle 120a can be slanted along the y-axis such that the tablet device retained in the receptacle 120a is not parallel to the z-x plane in order to secure the tablet device in position during different modes of operation (e.g., as a transporting entity carries the VR kit 100 between locations), shown in FIG. 1C and described in greater detail below. The receptacle 120a can also include a grip 121a at the entrance into the receptacle 120a or on one or more of its interior surfaces to retain the tablet device in position. During a mode of operation, described in greater detail below, the tablet device can protrude from the receptacle 120a so that the tablet device can be connected to a charging component (e.g., a power cord) during a mode of operation (e.g., storage). In other operation modes (e.g., during a transportation mode), the tablet device can be recessed within the receptacle 120a to protect the tablet device.

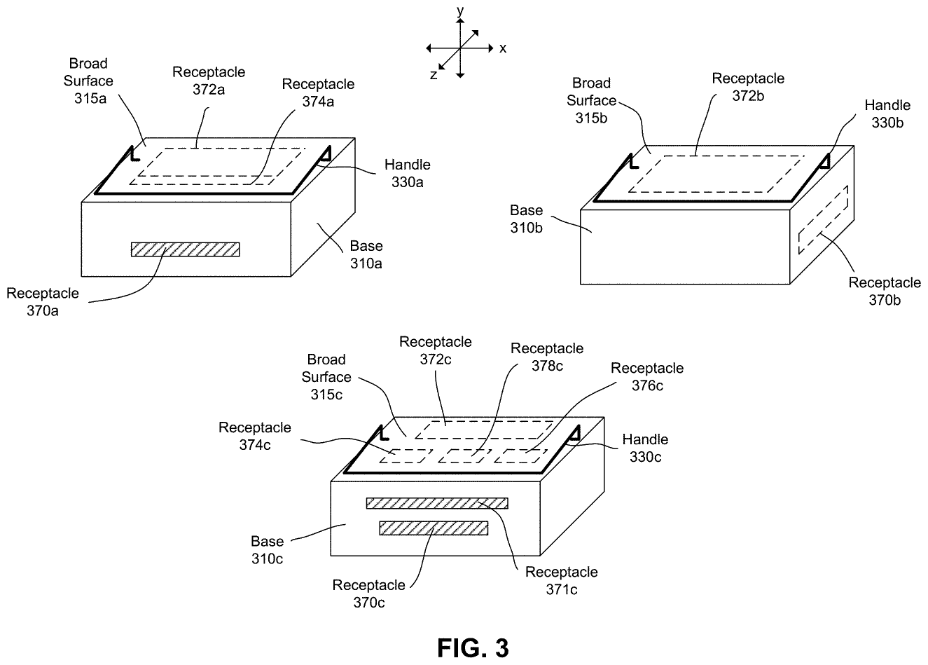

The VR kit 100 can additionally or alternatively include one or more receptacles (e.g., receptacle 120b, receptacle 120c) located on an upper broad surface 115 of the base 110 shown in FIG. 1A. The broad surface 115 is parallel to the x-z plane. The receptacles (e.g., 120b, 120c) are recessed within the broad surface 115 so that they can retain a component of a VR system. For example, the receptacle 120b can be recessed within the broad surface 115 and shaped so that a VR headset fits comfortably within the receptacle 120b. The receptacle 120b can be shaped so that the VR headset can be stored right side up or upside down orientation, while maintaining the integrity and functionality of the VR kit 100. The receptacle 120c can be shaped to retain a controller or a charging element. In alternative embodiments, each of the receptacles can be designed to retain any component of a VR system or other relevant components such as headphones, an audio system, sanitization wipes, etc. In alternative embodiments, the base 110 can include fewer or greater receptacles than the amount shown in FIG. 1A, and the receptacles can be positioned on any surface of the base 110, described in greater detail below in relation to FIG. 3A-3C. For instance, the base 110 can include another receptacle configured to store folders or other papers of a health care provider (e.g., nurse, doctor) transporting the VR kit 100 between locations.

FIG. 1B is a front view of the VR kit 100 shown in FIG. 1A. The receptacles (e.g., 120b, 120c) can have a depth in the y-direction, shown in FIG. 1B, such that the components are recessed within the base 110 without risk of falling out during transportation. The receptacles (e.g., 120b, 120c) are deep enough so that the handle 130 can move over the components (e.g., VR headset, controller) when they are in their respective receptacles without interfering with the components. Additionally, the handle 130 can have a configuration that, when the handle 130 is rotated about an axis, provides clearance with the components (e.g., when the handle is in an upright position shown in FIG. 1B). The receptacles are arranged with respect to each other and the handle 130 so that a proximity sensor in the VR headset is not blocked by the components, other portions of the base 110, the handle 130, or a user carrying the VR kit 100. The proximity sensor functions to facilitate automatic transition of the VR headset between an active mode and an inactive mode (e.g., off mode, idle mode) where, if the sensor is blocked, the VR headset may be unintentionally activated in and drain the battery while not in use. As such, the structural configuration of the base 110 prevents unintentional activation of the VR headset in coordination with the proximity sensor of the VR headset.

The handle 130 allows a user to carry the VR kit 100 in addition to other items that the user may be carrying. The handle 130 is adjustable so that the VR kit 100 can be stored compactly and so that a user can carry the system without compromising any of the components of the VR system. FIG. 1A shows the handle 130 in a compact position for storage and FIG. 1B shows the handle 130 in a mode suited for transportation. In FIG. 1B, a contact position 135 of the handle 130 (e.g., where a user touches the handle to carry the VR kit 100), is parallel with the x-axis such that the contact position 135 is aligned with a center of gravity of the VR kit 100 when the components (e.g., VR headset, controller) are retained in their respective receptacles. The user may grasp the handle at or near the midpoint along the contact position 135. According to the embodiment of FIG. 1B, the contact position 135 is co-planar with the center of gravity in a plane parallel to the x-y plane. This allows the VR kit 100 to maintain a balanced position during transportation. In this configuration, the broad surface 115 of the base 110 may be approximately parallel to the ground during one or more modes of operation.

FIG. 1C shows a side view of the embodiment of the VR kit 100 shown in FIG. 1B. A side of the base 110 shown in FIG. 1C is in a plane parallel to the y-z plane where the x-axis extends out of the page (e.g., towards the reader). The handle 130 is angled so that the contact position 135 is aligned with a center of gravity 101 of the VR kit 100. In more detail, the center of gravity 101 is the center of gravity 101 when the components of a VR system are retained in their respective receptacles. The handle 130 is configured so that the handle 130 cannot rotate beyond the center of gravity 101 (e.g., such that the contact position 135 is to the right of the center of gravity 101 in FIG. 1C). In one embodiment, the range of motion (shown by the arrow in FIG. 1C) of the handle 130 is limited to 80 degrees of rotation about an axis parallel to the x-axis.

FIG. 1D is an isometric view from the top right of the VR kit 100, according to one or more embodiments. The base 110 is approximately a square prism, where each side is approximately 6-12 inches wide. The receptacles (e.g., 120a, 120b, 120c) are shaped to retain specific components of a VR system. Receptacle 120a is configured to retain a tablet device. The width of receptacle 120a is approximately the width of a tablet device (e.g., 6-9 inches). Receptacle 120b is configured to retain a VR headset, shown by the shape of the receptacle 120b (e.g., the VR headset mates with the receptacle 120b). The receptacle 120b is shaped to prevent a VR headset from being damaged during storage or transportation. Receptacle 120c is configured to retain a controller for the VR headset. Receptacle 120c is deep enough (e.g., in the y-direction) so that the controller does not block the proximity sensor of the VR headset when the controller and the VR headset are retained in position.

Because medical personnel (e.g., a doctor, a nurse) may be transporting the VR kit 100 around a hospital, the base 110 can be composed of a lightweight material. In some embodiments, the base 110 is composed of a plastic material shell, where features (e.g., recesses for various receptacles) are formed in the shell. Alternatively, in some embodiments, the base 110 is composed of a material with a relatively low density, such as foam, and the base 110 is encapsulated by a shielding material, such as plastic. The shielding material may be used to protect internal components (e.g., an electronic subsystem) from the external environment (e.g., water, dirt, sanitizing agents). The base 110 and/or the shielding material can be a material that is compatible with sanitization products such as alcohol wipes, disinfectant sprays, and/or other common sanitization agents. As such, the base 110 can be highly resistant to corrosion. Furthermore, the base 110 can be durable enough so that it can be dropped or accidentally bumped without breaking or deforming. It also can have a strength and durability to support other VR kits 100 or items that may be stacked on top of the VR kit 100.

The receptacles (e.g., 120a, 120b, 120c) can be molded into the base such that the base 110 is a continuous piece. In other embodiments, the base 110 can be multiple pieces joined by an adhesive. The receptacles may also include a lining material (e.g., a cushioning material) so that delicate components of a VR system (e.g., optic glasses of a VR headset) are not broken, damaged, or scratched during one or more modes of operation. Similarly, the receptacles can include a lining material (e.g., a material with high coefficient of friction such as grip tape) for retaining the components of a VR system in position so that components do not move within their respective receptacle. In other embodiments, the receptacles can include any other materials or components useful for retaining components of a VR system in position.

The handle 130 shown in FIG. 1D is in a position configured for storage. In other embodiments, the handle 130 can be rotated about an axis parallel to the x-axis to a position configured for transportation. Furthermore, the handle 130 can be configured such that a user holding the VR kit 100 can hold other items (e.g., for use in a clinical setting). For instance, the handle 130 can allow an operator or other entity to hold other objects such as medical charts, medicine, etc. while still being able to transport a VR kit 100. However, as described above, the base 110 can additionally or alternatively include one or more receptacles for the other objects (e.g, patient medicine, charts, etc.), such that the operator does not have to carry other objects while transporting the VR kit 100.

In one embodiment, a user can grip the handle 130 with two fingers while supporting a weight of the VR system and VR kit 100. In one embodiment, the weight of a VR kit 100 with components of a VR system retained in their respective receptacles is approximately 4 pounds. The handle 130 can be composed of a strong material, such as aluminum or stainless steel, to support the weight of the VR kit 100 during transportation. Alternatively, the handle 130 can be composed of a metal, composite, polymer, etc. suitable for supporting the weight of the VR kit 100. It may also be composed of a material that is resistant to corrosion, particularly in relation to sanitizing agents. In alternative embodiments, the handle 130 can be composed of any suitable material for interacting with the VR kit 100 and a VR system.

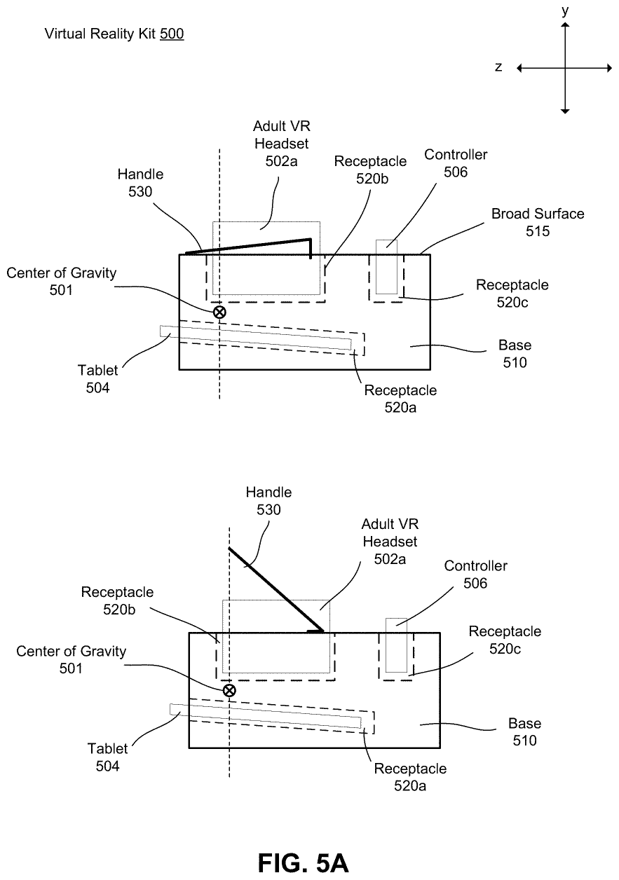

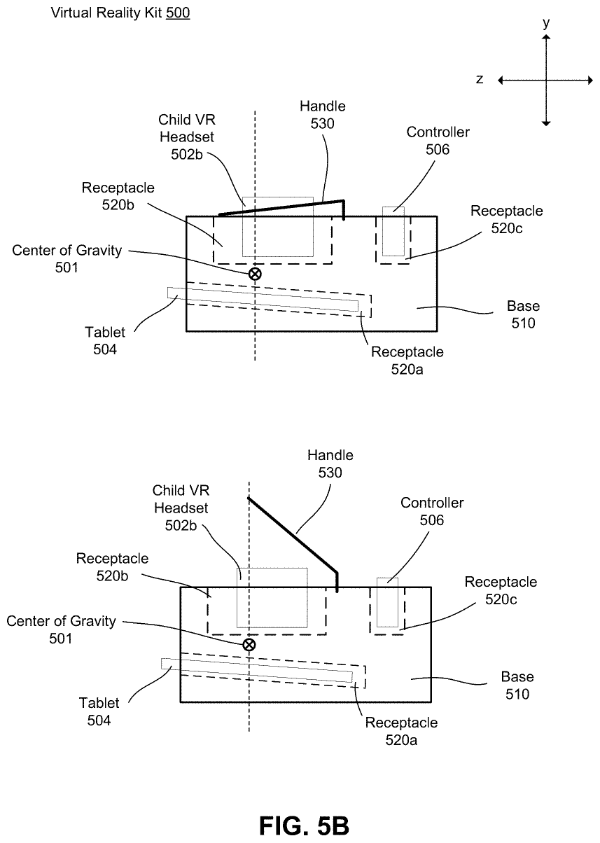

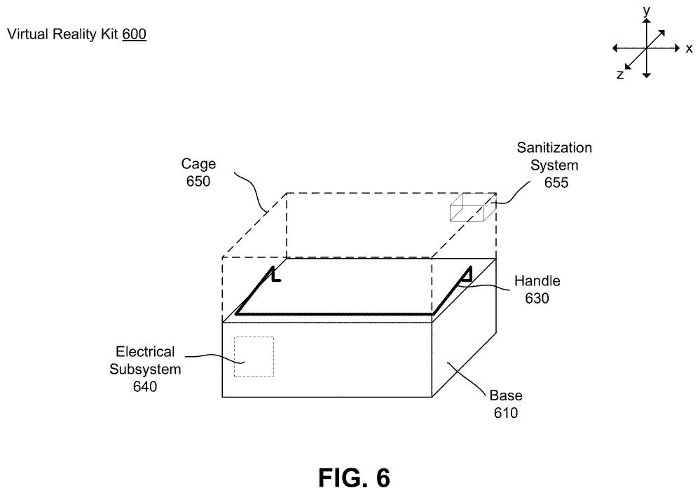

In some embodiments, the position or orientation of the handle 130 can be adjusted to remain in line with the center of gravity 101 of the VR kit 100 if the weight of the VR kit 100 changes, as described in greater detail below in relation to FIGS. 5A-5B. The VR kit 100 can also include additional components, described in greater detail below in relation to FIGS. 6-8. The additional components can include an electrical subsystem, a cage, a sanitization system, and/or other components relevant to a VR system.

The virtual reality kit 100 thus combats problems with using virtual reality systems on a wide spread scale, particularly in clinical settings. As described above in relation to FIG. 1A-1D, the VR kit 100 and its components have a compact design that allows for easy storage (e.g., of multiple units in a small space). The VR kit 100 can also be easily carried by a medical personnel, who may be carrying other items such as charts and other medical equipment. The system is lightweight and in one embodiment, a user can carry the VR kit 100 with two fingers without putting extensive strain on his/her hands. The VR kit 100 does not require a patient to travel in order to use the VR system. Furthermore, the VR kit 100 facilitates the transition between users by easing the sanitization process of a VR system as the VR kit 100 may be composed of materials that are compatible with sanitizing agents.

### 2. Base Variations

FIG. 2 illustrates a first (top left), second (top right), third (bottom left), and fourth (bottom right) embodiment of a base of a VR kit, according to one or more embodiments. In the embodiment of FIGS. 1A-1D, the base 110 is a rectangular prismatic in form factor so that the VR kit 100 can be stored compactly. The embodiments of FIG. 2 illustrate other configurations of a base that can be compatible with a VR system. FIG. 2 shows four isometric views of different embodiments of a base, where a front side of each base is parallel to the x-y plane and the z-axis is extending towards the reader.

In the first embodiment (top left), a base 210a is cylindrical prismatic in form factor. The embodiment of the handle 230a has a concave profile such that the handle 230a can mate with a broad surface 215a of the base 210a; however, as described in further detail below, the variations of the base embodiments and the handle embodiments can be combined in any other suitable manner. The cylindrical base 210a can allow for stability of a VR kit during transportation. In the first embodiment, the cylindrical base 210a has diameter that is larger than its height (e.g., the base 210a is wider than it is tall). In other embodiments, the base 210a can have a diameter that is smaller than its height. As such, components of a VR system may be stored in alternative orientations (e.g., a VR headset may be stored in a sideways direction so that eyeglasses of the VR headset are approximately parallel with the y-axis). This may allow for a more compact storage arrangement of a VR kit.

The second embodiment (top right) shows a parallelepiped base 210b. In one embodiment, the base 210b may be a parallelepiped so that multiple bases 210b can be oriented side by side (e.g., a first side surface 217b of a first base 210b is coupled to a second side surface 218b of a second base 210b). As such, two or more bases 210b can mate in order to minimize space occupied by the bases 210b. The base 210b can also be shaped so that the weight of the kit is balanced when components of a VR system are stored in their respective receptacles. A contact position 235b of the handle 230b is shown to be parallel with the x-axis. In alternative embodiments, the handle 230b can be rotated 90 degrees about an axis parallel to the y-axis (e.g., the contact position 235b is parallel to an edge of the second side surface 218b of the base 210b). Furthermore, the handle 230b can be located in a position to align with a center of gravity of the base 210b, as described above in relation to FIG. 1C.

The third embodiment (bottom left) of FIG. 2 illustrates a base 210c with a trapezoidal cross section (e.g., in an x-y plane as shown in FIG. 2, bottom left). The base 210c can be wider at the bottom (e.g., a surface opposing a broad surface 215c has a larger area than the broad surface 215c) so that it is stable during storage and transportation. In some embodiments, the broad surface 215c of the base 210c is only slightly smaller than its opposing surface. In other embodiments, a width of the broad surface 215c may be significantly smaller than a width of its opposing surface of the base 210c. A handle 230c can be shaped to align with the broad surface 215c of the base 210c in a first position.

The fourth embodiment (bottom right) shows a base 210d with a rectangular cross section with rounded edges. The rounded edges of the base 210d may provide a safer operational system for the user and/or patient. In the fourth embodiment, all four edges of the cross section are rounded. In alternative embodiments, some of the edges may be sharp and some may be round. Shown in the fourth embodiment, a sharp edge of a handle 230d can also be rounded to mate with a broad surface of 215d of the base 210d.

FIG. 2 thus illustrates several embodiments of a base used in a VR kit. In alternative embodiments, the base of a VR kit can have any shape suitable for supporting a VR system. The base of the VR kit base (e.g., 210a, 210b, 210c, 210d) can include a plurality of receptacles, described in greater detail in relation to FIG. 3, for retaining components of a VR system. For simplicity, receptacles are not shown in FIG. 2. Furthermore, in alternative embodiments, the base of the VR kit base (e.g., 210a, 210b, 210c, 210d) can also be hollow so that components of a VR system can be retained within an internal cavity of the base.

### 3. Receptacle Variations

In the embodiment of FIGS. 1A-1C, the base 110 includes three receptacles. In alternative embodiments, the base 110 can include fewer or more receptacles, described in greater detail below. The receptacles can also be oriented in a different manner than described above and can be configured to retain different components than described above. FIG. 3 illustrates three embodiments of a VR kit, according to one or more embodiments. The embodiments of FIG. 3 each shows an isometric view of a VR kit from the top right, where a front of a base of a VR kit is parallel with the x-y plane.

The first embodiment (top left) of FIG. 3 includes a base 310a with three receptacles. Receptacle 370a is configured to receive a tablet device, as described above. The base 310a can be stored so that the receptacle 370a faces towards a user (e.g., the receptacle 370a is located on a front surface of the base 310a). Receptacle 372a and receptacle 374a are located on a broad upper surface 315a of the base 310a. Receptacle 372a can be configured to retain a VR headset and receptacle 374a can be configured to retain a controller for the VR headset. In one embodiment, receptacle 374a is recessed further in the base 310a in the y-direction than receptacle 372a so that the controller is housed below the broad surface 315a and the controller does not block a proximity sensor of the VR headset, as described above. In the first embodiment (top left), receptacle 372a is located on the broad surface 315a opposite from a resting position handle 330a (e.g., the handle 330a does not rotate over receptacle 372a).

The second embodiment (top right) includes a base 310b with two receptacles. Receptacle 370b is located on a side of the base 310b. The receptacle 370b can be configured to retain a tablet device or other component of a VR kit. In one embodiment, the receptacle 370b has a door mechanism, so that a tablet device can be recessed within the receptacle 370b and the receptacle 370b can be sealed from the external environment. The base 310b can thus be stored compactly in any orientation. In some embodiments, the door mechanism can be a sliding door that slides along an axis parallel to the z-axis (e.g., slides towards/away from a user who may be facing the front of the VR kit). The door mechanism can also be attached by a hinge or other coupling mechanism. The base 310b also includes a receptacle 372b on the broad surface 315b. A surface area of the receptacle 372b is slightly smaller than a surface area of a broad surface 315b and is configured to retain at least a VR headset. The receptacle 372b can also retain other components such as headphones, a charging cord, a controller, etc. The receptacle 372b can include removable walls or barriers (not shown) so that each component of a VR kit can be retained in a different compartment within the receptacle 372b. The walls can be adjustable so that a user can choose to transport certain components of a VR system at one time.

The third embodiment (bottom middle) illustrates a base 310c with six receptacles. Receptacle 370c and receptacle 371c are located on the front of the base 310c. Receptacle 370c is configured to retain a tablet device, while receptacle 371c is configured to retain other items relevant to a patient or medical personnel (e.g., charts, medical records, liability forms, directions for the VR kit, etc.). In some embodiments, receptacle 371c is wider than receptacle 370c. Alternatively, the receptacles can be any suitable width for retaining components of a VR kit or other relevant materials. The base 310c includes four receptacles on a broad surface 315c. The receptacles (e.g., 372c, 374c, 376c, 378c) can be configured to retain components of a VR system and/or other relevant items including a VR headset, a controller, a set of headphones, a charging cable, sanitizing wipes, a battery, an adjustable head strap, patient medicine, etc. Each of the receptacles can be configured to retain any of the items, providing that a handle 330c is able to have a range of motion that clears each of the components retained in the receptacles. The receptacles are also arranged such that components retained in the receptacles do not block a proximity sensor of a VR headset.

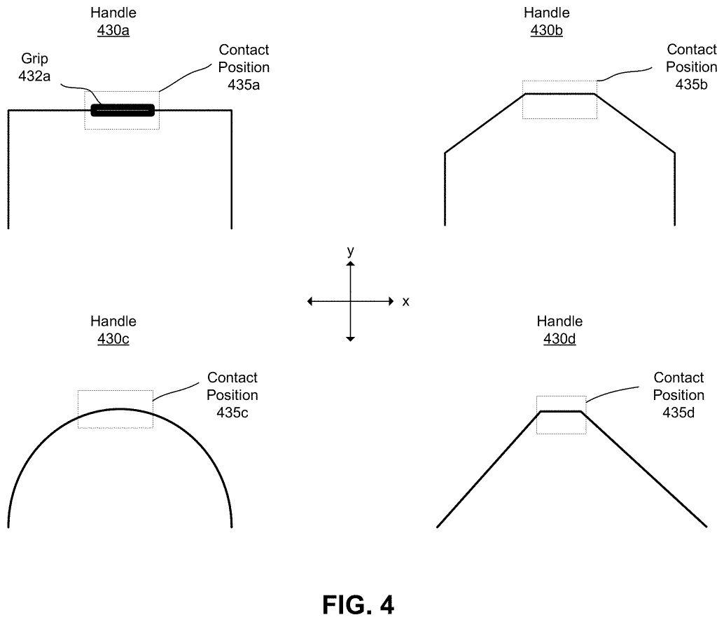

FIG. 3 thus shows several embodiments of a VR kit including a plurality of receptacles. The embodiments shown in FIG. 3 are not exclusive or exhaustive of the orientation of receptacles in a VR kit. Furthermore, each embodiment shown in FIG. 3 can be combined with any of the embodiments of bases shown in FIG. 2. The embodiments in FIG. 2 and FIG. 3 can also be combined with different versions of a handle, described below in relation to FIG. 4. The receptacles are configured with a handle so that the handle can rotate from a first position (e.g., for storage) to a second position (e.g., for transportation) without interfering with any of the components retained in position by the receptacles. Finally, in some embodiments, a base (e.g., 310a, 310b, 310c) can include receptacles located on a surface opposing a broad surface (e.g., 315a, 315b, 315c) so that two or more VR kits (e.g., 100) can be stacked on top of each other. As such, components (e.g., VR headset, controller, handle) protruding from a broad surface of a first VR kit can mate with the receptacles on an opposite surface of a broad surface of a second VR kit stacked on top of the first VR kit.

### 4. Handle Variations

FIG. 4 illustrates a first (top left), second (top right), third (bottom left), and fourth (bottom right) embodiment of a handle of a VR kit, according to one or more embodiments. Each of the embodiments shows a handle that could be attached to various embodiments of a base. Furthermore, as described below, variations of the handle components, contact positions, and/or grip elements can be combined in variations of the handle not depicted in FIG. 4. The handle can be composed of a material (e.g., stainless steel, plastic, etc.) that is easily sanitized and does not degrade or corrode under different sanitization methods; however, the handle can alternatively be composed of other materials.

The first embodiment (top left) of handle 430a includes a handle 430a with two side components parallel to the y-axis shown in FIG. 4 and a horizontal component parallel to the x-axis and connecting the side components. In some embodiments, the side components may not be vertical, as they may be angled so that the horizontal component is aligned with a center of gravity of a VR kit, described in greater detail below in relation to FIGS. 5A-5B. The handle 430a includes a contact position 435a so that a user can interact with the handle 430a. The contact position 435a can include a grip 432a in order to provide user comfort. The grip 432a can be composed of an elastic material such as rubber so that a user can comfortably hold the grip 432a. In other embodiments, the grip 432a may be another material (e.g., plastic) shaped to mate with a user's hand and/or fingers.

The second embodiment (top right) includes a handle 430b with five components. Two of the components (e.g., the sides) are parallel with the y-axis. In some embodiments, the sides may not be vertical, as they may be angled so that the horizontal component is aligned with a center of gravity of a VR kit. An angled component extends from a free end of each of the side components at approximately a 45 degree angle, and the angled components are connected by a horizontal component parallel with the x-axis. The horizontal component can be configured to interact with a user. The horizontal component may be a contact position 435b, where the contact position 435b has a width approximately equal to the width of a hand of a user (assuming the user has a hand of average size). In alternative embodiments, the width of the contact position 435b may be approximately equal to the width of two fingers of a user (e.g., pointer finger and middle finger). The handle 430b can be composed of a rigid material so that the handle 430b maintains its position during different modes of operation.

The third embodiment (bottom left) shows a handle 430c with a concave cross section. The handle 430c can have a concave cross section to mate with a cylindrical base (e.g., the base 210a in the first embodiment in FIG. 2) or any other suitable base form factor. In the third embodiment (bottom left), the handle 430c can include a contact position 435c for the user to hold the handle 430c. In one embodiment, the contact position 435c may rounded so that a user can comfortably interact with the handle 430c. Alternatively, the handle 430c can be composed of a flexible material (e.g., like a material of a handle attached to a beach bucket) so that the handle 430c can bend to provide user comfort during use while supporting the weight of a VR kit.

The fourth embodiment (bottom right) illustrates a handle 430d with a nearly triangular cross section. The handle 430d has two angled components, joined by a horizontal component. The horizontal component is a contact position 435d. Similar to the second embodiment (top right), the width of the contact position 435d can be approximately the width of two fingers of an average user or approximately the width of hand of an average user. Alternatively, the contact position 435d can be any suitable width for interacting with a user.

As described above, the embodiments shown in FIG. 4 are not exclusive or exhaustive of handles that can be included in a VR kit. For example, a circular handle shown in the third embodiment (bottom left) can include a grip shown in the first embodiment (top left). Each of the handles described in FIG. 4 can be configured to interact with a base, including the bases described by FIG. 2. For example, the second embodiment (top left) in FIG. 2 can be combined with the first embodiment (top left) of FIG. 4 to create a VR kit. In one embodiment, a handle (e.g., handles described in relation to FIG. 4) can be adjusted so that the contact position of the handle is aligned with the center of gravity of a VR kit, as described below in relation to FIGS. 5A-5B.

FIG. 5A illustrates a first handle position (top) and a second handle position (bottom) of a first embodiment of a VR kit 500, according to one or more embodiments. The VR kit 500 is substantially the same as VR kit 100. FIG. 5A shows a side view of a VR kit 500, where a side shown is parallel to the z-y plane and the x-axis is extending out of the page. The VR kit 500 includes a base 510, receptacles 520a, 520b, 520c, and a handle 530. Receptacle 520a retains a tablet 504, receptacle 520b retains an adult VR headset 502a, and receptacle 520c retains a controller 506. The tablet 504 is protruding from receptacle 520a so that the tablet can be easily connected to a power cord and/or other external wire. The tablet 504 can also be easily removed from receptacle 520a by a user. The handle 530 can move from a first positon (top) where a contact position of the handle 530 is coupled to a broad surface 515 of the base 510, to a second position (bottom), where the contact position is aligned with the center of gravity 501. In one embodiment, the center of gravity 501 is the center of gravity 501 of the VR kit 500 when the tablet 504, controller 506, and adult VR headset 502a are retained in their respective receptacles. In alternative embodiments, the center of gravity 501 can be a position when different and/or additional components are retained in one or more of the receptacles. The handle 530 can also include a locking mechanism that prevents the handle 530 from rotating beyond the desired position (e.g., the handle 530 does not rotate to a position where the contact position of the handle 530 is behind/to the right of the center of gravity 501). In the embodiment shown in FIG. 5A, a portion of the handle 530 nearest the base 510 includes an angled feature that functions as a stop, such that the stop maintains alignment of the contact position of the handle 530 when the VR kit 500 is being carried by the handle.

In some embodiments, it may be useful to adjust the handle 530 so that the handle 530 can maintain alignment with the center of gravity 501 when alternate components are retained in the receptacles or when components are missing from their respective receptacles. FIG. 5B illustrates a first handle position (top) and a second handle position (bottom) of a second embodiment of the VR kit 500. In the embodiment of FIG. 5B, a child VR head set 502b is retained in receptacle 520b, rather than the adult VR head set 502a as shown in FIG. 5A. The child VR head set 502b may weigh less than the adult VR head set 502a and/or have a different mass distribution because it is smaller and is configured to interact with a smaller user. When the child VR headset 502a is retained in the receptacle 520b, the center of gravity 501 of the VR kit 500 may thus be affected. A new position of the center of gravity 501 may be to the right of the old position (e.g., the center of gravity 501 translates in the negative z-direction). In alternative embodiments, the center of gravity 501 can translate in the positive z-direction (e.g., when a heavier tablet 504 is recessed in receptacle 520a, when the controller 506 is removed from receptacle 520c, etc.). Because the center of gravity 501 has a new position due to the child VR headset 502a, the handle 530 may no longer be aligned with the center of gravity 501 in its second position (e.g., an upright position for transportation). The handle 530 can thus include an adjustment mechanism that allows the position of the handle 530 to translate so that it is aligned with the new center of gravity 501 in the second position. FIG. 5B shows the first position (top) and the second position (bottom) of the handle 530 when it is realigned with the center of gravity 501 due to the change in weight of the VR kit 500.

In alternative embodiments, the weight of the VR kit 500 may be adjusted for another purpose and the handle 530 can be adjusted accordingly. For example, if the controller 506 is removed from its position, the center of gravity 501 may shift. As such, the handle 530 can be adjusted by the adjusting mechanism to maintain alignment. Alternatively, the base 510 can include an adjustable counterweight, so that the weight of the VR kit 500 can be adjusted so that the handle 530 can maintain alignment with the center of gravity 501 without adjusting the handle 530. The center of gravity 501 can also translate along an axis parallel to the y-axis. In the embodiments of FIGS. 5A-5B, translation of the center of gravity 501 along an axis parallel to the y-axis is not relevant to the position of the handle 530 in line with the center of gravity 501. However, in alternative embodiments, the VR kit 500 can include an adjustment mechanism for aligning the center of gravity 501 along another axis. In alternative embodiments, the VR kit 500 can include additional elements for maintaining alignment of the handle 530 with the center of gravity 501.

### 5. Additional Components

FIG. 6 illustrates an isometric view from the top right of a VR kit 600, according to one or more embodiments. The virtual reality kit 600 is substantially similar to VR kit 100 with additional components. VR kit 600 includes a base 610, a handle 630, an electrical subsystem 640, a cage 650, and a sanitization system 655. The VR kit 600 also includes receptacles (not shown) for retaining components of a VR system.

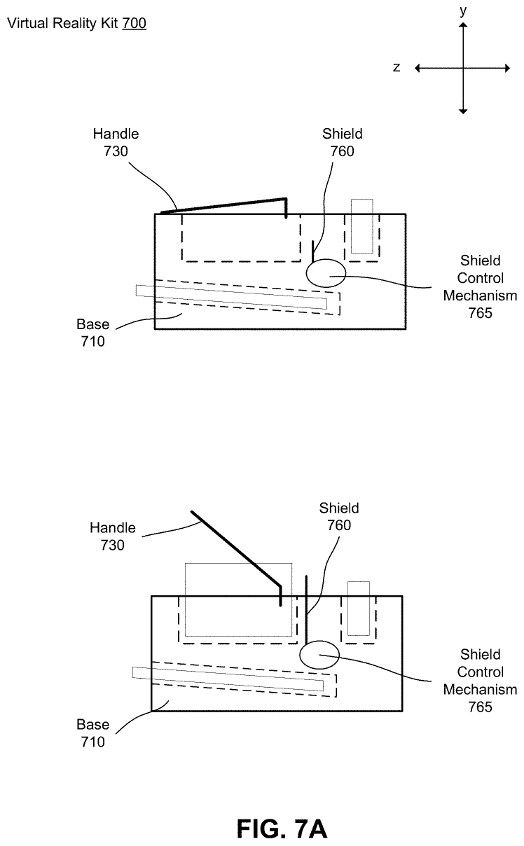

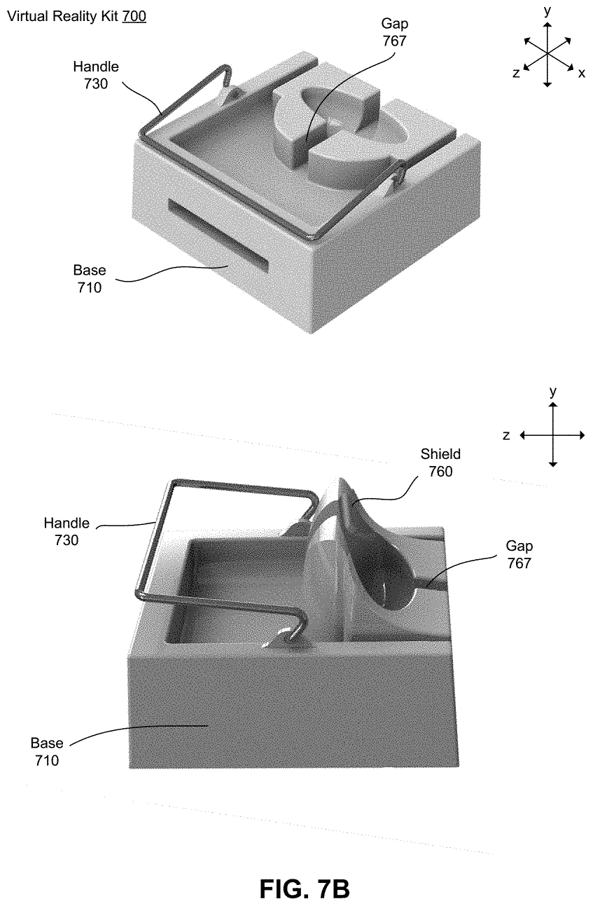

The electrical subsystem 640 can be configured to interact with components of a VR system and/or external components (e.g., a computer). In one embodiment, the electrical subsystem 640 is a battery. In other embodiments, the electrical subsystem 640 is another component of a power system (e.g., a VR headset is charged via the electrical subsystem 640). The base 610 can include a port for connecting the base 610 and/or the electrical subsystem 640 to an outlet via a power cord. Alternatively, a VR system can be charged wirelessly (e.g., by inductive charging). Components of the VR system can be charged regardless of the orientation of the VR system within the base 610 (e.g., the VR headset can be placed upside down or right side up). In some embodiments, such as in FIG. 7B, the base 610 includes a gap to retain and/or guide a wire for charging. The base 610 or the electrical subsystem 640 can include a light indicating the battery level of a component of a VR system (e.g., red light indicates low power). The electrical subsystem 640 can also be used to power a sanitization system 655, described below.

In other embodiments, the electrical subsystem 640 can be another electrical component configured to interact with a VR system retained in the VR kit 600. For example, the electrical subsystem 640 can be a processor or other type of analysis engine that receives signals and/or information from a VR headset. As such, the electrical subsystem 640 can be configured to transmit information to a computer via an external cord or a network. The electrical subsystem 640 can also include a display element that broadcasts an image feed produced on the VR headset while a patient is using the VR headset so that a person not using the VR headset (e.g., a doctor, a nurse) can view the same screen as a patient. A display element may also provide information related to the VR system, a patient, or other relevant data such as environmental conditions. Furthermore, the electrical subsystem 640 can be an audio system or a microphone that can interact with a VR headset and/or a patient. In alternative embodiments, the electrical subsystem 640 can be any electrical component relevant to a VR kit 600.

One or more embodiments of the VR kit 600 may include a cage 650. The cage 650 may be optional during different modes of operation (e.g., in use during storage, removed during transportation). The cage 650 can function to protect components of a VR system, or it may be used for storage purposes (e.g., the cage allows for stacking of VR kits 600). The cage 650 may be an independent component from the base 610 or it may be coupled to the base 610 via a hinge or other attachment mechanism. In FIG. 6, the cage 650 includes a sanitization system 655. In one embodiment, the sanitization system 655 is a UV light that turns on when the cage 650 is closed. In other embodiments, the sanitization system 655 is a mechanism for spraying disinfectant. A UV light and/or a spraying mechanism can be powered by the electrical subsystem 640. Alternatively, the sanitization system 655 can be any other sanitization agent that is compatible with the VR kit 600 and a VR system, or it can be included in another component of the virtual reality kit 600.

In some embodiments, the virtual reality kit 600 can be configured to interact with existing medical systems such as an IV station or a crash cart. For example, the handle 630 can be hooked onto an IV station or a crash cart. The virtual reality kit 600 can also be configured to transport or store other systems such as biometric sensor systems or non-medical electronic systems. In some embodiments, the electrical subsystem 640 can interact with other medical equipment.

FIGS. 7A-7B depict configurations of a shield for protecting lenses of a head mounted display, where the shield is optionally transitionable between different configurations, according to one or more embodiments. In more detail, FIGS. 7A-7B each show a first position of a shield (top) and a second position of a shield (bottom) of an embodiment of a VR kit 700, according to one or more embodiments. The embodiments shown in FIGS. 7A-7B include a base 710, a handle 730, and a shield 760 for shielding a VR headset from light. In some embodiments, optical elements (e.g., lenses) of a VR headset may be sensitive to light. Extensive exposure to light can have a negative effect on the lifetime and/or performance of a VR headset. The VR kit 700 includes a shield 760 so that the optical elements of the VR headset can be protected from long term exposure to light.

FIGS. 7A-7B illustrate a first position (top) and a second position (bottom) of the shield. In FIGS. 7A-7B, the shield 760 is adjustable by a shield control mechanism 765. The shield control mechanism 765 maintains the shield 760 in a recessed position when a handle 730 is in a first position (e.g., handle 730 is not in use). In the first position (top), it is assumed that a VR headset and other system components may be removed from their receptacle for user operation. The shield 760 is transitioned to second position (bottom) by the shield control mechanism 765 when the handle 730 is in an upright position (e.g., when the VR kit 700 is being transported, and thus, the VR headset is not in use). The shield 760 is positioned so that it blocks an optic component of a VR headset from light but does not block a proximity sensor of a VR headset.

In the embodiment shown in FIG. 7B, the VR kit 700 includes a gap 767 such that the proximity sensor is not blocked regardless of the orientation of the VR headset (e.g., the VR headset can be stored upside down or right side up). The gap 767 is located so that the lenses of the VR headset are blocked by the shield 760 but the proximity sensor is not during a mode of operation. In some embodiments, the handle 730 operates in conjunction with (e.g., controls) the shield control mechanism 765. The shield control mechanism 765 can alternatively be powered by an electrical subsystem (e.g., electrical subsystem 640). In alternative embodiments, the shield 760 can be in an operational position when the handle 730 is in a first position (e.g., when the VR kit 700 is not stored in a dark location the shield 760 may be useful for blocking light). In one example, it order to protect the lenses from light during charging, the shield 760 is raised responsive to plugging in the VR system and/or placing the VR system in charge mode.

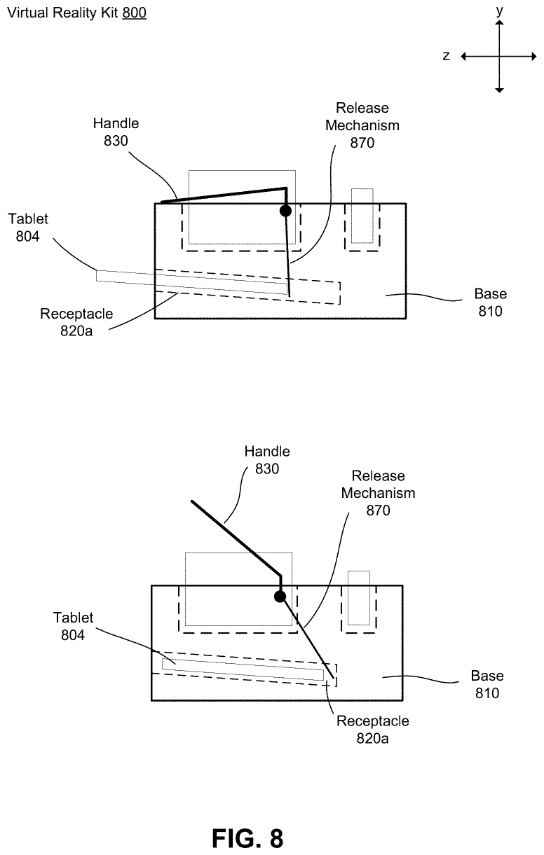

FIG. 8 illustrates a first position of a tablet (top) and a second position of a tablet (bottom) of an embodiment of a VR kit 800, according to one or more embodiments. The VR kit 800 is substantially the same as VR kit 100 with an additional component. VR kit 800 includes a release mechanism 870 configured to interact with a component of a VR system recessed in receptacle 820a. FIG. 8 shows a first position (top) and a second position (bottom) of a tablet 504 controlled by a release mechanism 870. The release mechanism 870 may be controlled by a handle 830. In the first position (top), the release mechanism 870 is in contact with the tablet 804 and the handle 830. The release mechanism 870 can act like a pendulum, such that when the handle 830 is in a first position (top), the tablet 804 is pushed out of its respective receptacle 820a by the release mechanism 870. This allows for a user to easily remove the tablet 804 from receptacle 820a. When the handle 830 is in a second position (bottom), the tablet 804 is recessed within the receptacle 820a so that the tablet 804 does not fall out during transportation. In one embodiment, the receptacle 820a can have a latch or door to seal the receptacle 820a from the external environment. The release mechanism 870 can also operate in conjunction with the door such that when the handle is in a first position (top), the door is opened and when it is in a second position (bottom) the door is closed. In alternative embodiments, a release mechanism 870 can include additional components, such as a spring, for releasing a component of a VR system from its respective receptacle. Additionally, a release mechanism 870 can be included or attached to another receptacle of the VR kit 800.

FIGS. 6-8 thus show additional components of a VR kit in different embodiments. Alternatively, components shown in FIGS. 6-8 could be combined into a variety of embodiments. For example, a VR kit 100 can include a release mechanism 870 and a cage 650. Alternatively, a VR kit 100 can include a sanitization system 655 and a shield 760. The components described in FIGS. 6-8 can also be configured with any of the bases, receptacles, and handles shown in FIGS. 2-4 to create a functioning VR kit. The VR kit 100 can also include additional components not described herein.

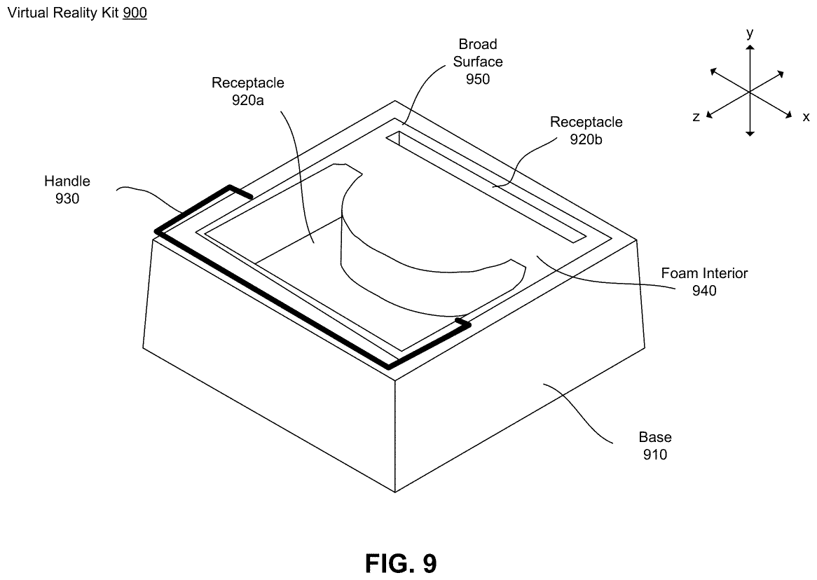

FIG. 9 illustrates an isometric view of the VR kit 900, according to one or more embodiments. The virtual reality kit 900 is substantially similar to VR kit 900 with additional components. VR kit 900 includes a base 910, a handle 930, and a foam interior 940. The base 920 includes an interior cavity in which components of a virtual reality system may be stored. The interior cavity is a hollowed region of the base 910 such that one face of the interior cavity is a bottom surface of the base 910 and the other face of the cavity is an exposed surface of the base 910. As illustrated in FIG. 9, the interior cavity of the base is filled with a foam interior 940. The foam interior 940 may be permanently positioned within the cavity of the base 910 or removably inserted into the cavity of the base 910. Sections of the foam 940 may be removed to create one or more receptacles (for retaining components of a virtual reality system in fixed positions. The foam interior 940 may also be configured to cushion or support components of a VR system to prevent the components from being broken, damaged or scratched during operation, storage or transportation of the VR system. Additionally, the foam interior 940 can be closed cell foam in order to keep germs out of the foam.

The foam interior 940 includes one or more receptacles (e.g. receptacles 920a and 920b) located on an upper broad surface 950 of the base 910 as shown in FIG. 9. The broad surface 950 is parallel to the x-z plane. The receptacles (e.g., 920a and 920b) are recessed within the broad surface 950 of the foam interior 940 so that they can retain components of a VR system. The receptacle 920a is substantially similar to the receptacle 120b. The receptacle 920a can be shaped so that the VR headset can be oriented and stored right side up or upside-down, while maintaining the integrity and functionality of the VR kit 900. The receptacle 920a is substantially similar in function to the receptacle 120b. The receptacle 920b is substantially similar in structure and function to the receptacle 120a in that the receptacle 920b is configured to receive a tablet device or to house another electronic device, such as a cell phone, used to communicate with a VR headset and/or store information related to a patient and/or a VR system. While the receptacle 120a is a recess in the front of a base, the receptacle 920b is a recess in the broad surface 950 of a foam interior. In addition to the receptacles 920a and 920b, the foam interior 940 may be configured to include additional receptacles (not shown).

### 6. Top Variations

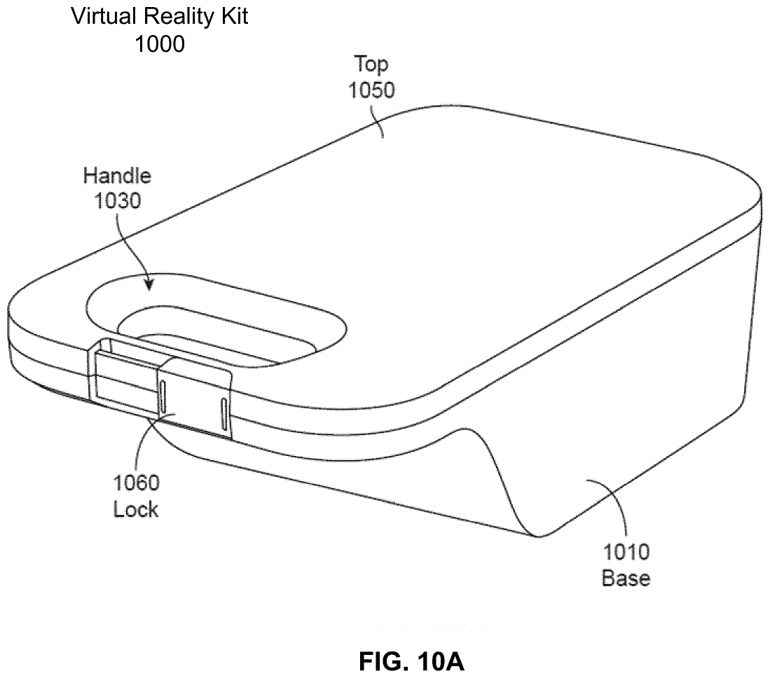

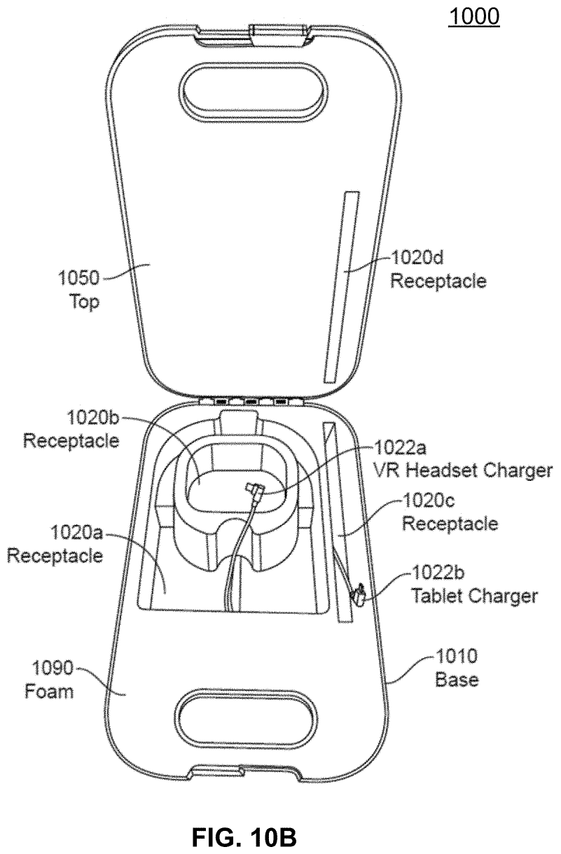

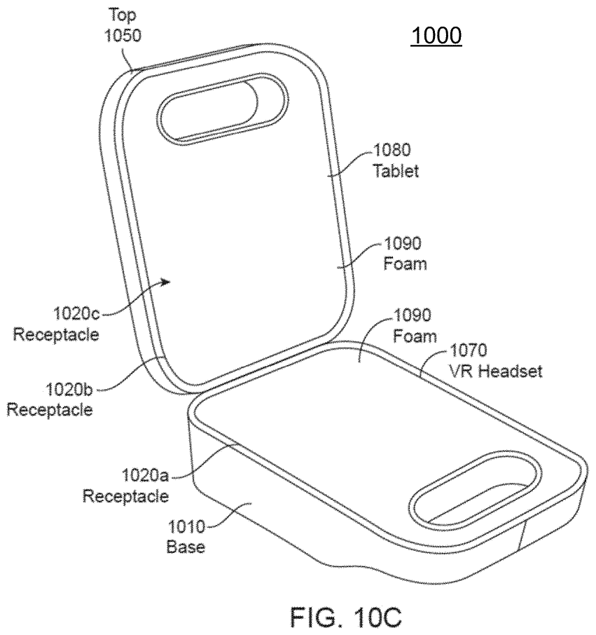

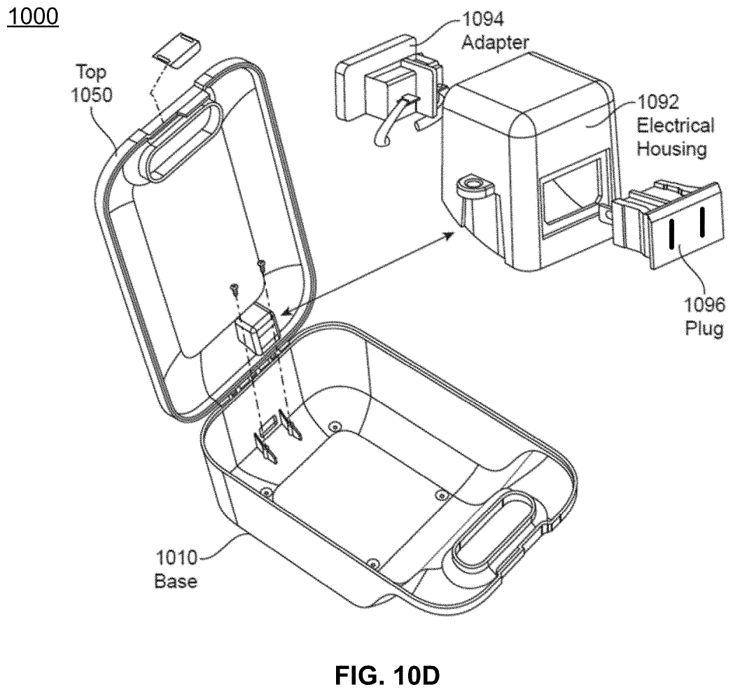

FIG. 10A illustrates an isometric view of a VR kit 1000, according to one or more embodiments. The VR kit 1000 is configured with a hinged casing to house components of a VR kit or alternate configurations of VR kits described herein, for example VR kit 900. The virtual reality kit 1000 comprises a base 1010, a handle 1030, a top 1050, and a lock 1060. The base 1010 is substantially similar to the base 110 and alternate configurations of the base described herein. The handle 1030 is functionally similar to the handle 130 and alternate configurations of handle described herein. In the illustrated embodiment of FIG. 10A, the handle 1030 is comprised of a first half that is part of the base 1010 and a second half that is part of the top 1050. When the top 1050 is coupled to the base 1010, the handle 1030 is completed such that a user may carry or transport to the VR kit 1000 without releasing the component of the VR system form the base 1010.

The top 1050 is a removable component of the VR kit 1000, configured to couple to the base 1010 to secure the components of a VR system stored in the VR kit 1000. In one embodiment, the entire perimeter edge of the top 1050 may be removed from the base 1010 to access components of a VR system stored in receptacles of the base 1010. In an alternate embodiment, the top 1050 may be rotated on a hinge along an edge of the base 1010 enable a user to access components stored within receptacles of the base 1010. The top 1050 may be designed using the same material as the exterior walls of the base 1010, a substantially similar material, or a different material. In some embodiments, the top 1050 may be designed with a durable material such that the exterior of the top 1050 may withstand damage while protecting components of a VR system stored in the base 1010.

When the top 1050 is coupled to the base 1010, a lock 1060 may hold the top 1050 in a position coupled with the base 1010. In the illustrated embodiment of FIG. 10A, a single lock 1060 is used to hold the top 1050 in position relative to the base 1010. However, in other embodiments, multiple locks 1060 may be used to hold the top 1050 in position.

FIG. 10B illustrates a front view of the VR kit 1000, according to one or more embodiments. In the illustrated embodiment, the top 1050 is rotated about a hinge coupled to the base 1010 to an open configuration. In a closed configuration (not shown), receptacles in the base 1010 may not be exposed to a user, however in the illustrated open configuration receptacles in the base 1010 are exposed for a user to access. The base 1010 includes multiple receptacles configured to securely store various components of a VR system. In the illustrated embodiment of FIG. 10B, the receptacle 1020a is configured to accommodate a VR headset of a VR system, a receptacle 1020b is configured to accommodate a controller of a VR system, and a receptacle 1020c is configured to accommodate a tablet of a VR system. The receptacle 1020c may alternatively be configured to securely store a different display device, for example a screen or a phone.

The receptacle 1020a is a cavity in the base 1010 designed to secure a VR headset during transportation of the VR kit 1000. The receptacle 1020a further comprises a VR headset charger 1022a. When stored or transported in the receptacle 1020a, the VR headset charger 1022 may be used to charge the VR headset. Similarly, receptacle 1020b may also be a cavity in the base 1010 designed to secure a VR controller during transportation of the VR kit 1000. The receptacle 1020c is a first recess in the base 1010 designed to secure a bottom portion of a tablet in place during transportation or storage of the VR kit 1000. Additionally, the receptacle 1020d is a second recess aligned with the receptacle 1020c along a vertical axis and designed to secure a top portion of a tablet during transportation or storage of the VR kit 1000. Accordingly, adjustment of the top 1050 from the open configuration to a closed configuration causes the receptacle 1020d to align with the receptacle 1020c to secure the entirety of the tablet. The receptacle 1020 further comprises a tablet charger 1022b. When stored in the receptacle 1020c, the tablet (or alternative display device) may be charged using the tablet charger 1022.

FIG. 10C illustrates an isometric view of the VR kit 1000 with a foam layer 1090, according to one or more embodiments. In the illustrated embodiment, the foam 1090 is designed to cover the entire surface area of the base 1010 without obstructing the grip of the handle 1030. The foam 1090 is further configured such that the top 1050 is able to seamlessly couple to the base 1010. When the top 1050 is locked in place relative to the base 1010, the foam 1090 further secures the position of components in the receptacles 1020. Additionally, the foam 1090 provides a cushioned support to prevent damage to stored components of the VR system during transportation of the VR kit 1000.

FIG. 10D illustrates an isometric view of the VR kit 1000 with an exploded view of electric components integrated into the VR kit 1000, according to one or more embodiments. The top 1050 includes an electrical housing 1092 coupled to an interior face of the base 1010. The electrical housing 1092 has two openings on opposite faces of the housing 1092. The first opening is designed to receive an electrical plug 1096 and the second opening is designed to receive an adapter 1094. Accordingly, an external plug may be connected to the electrical housing through the base 1010 to charge various components of a VR system stored within the VR kit 1000. The adapter 1094 may be used to accommodate the VR headset charger 1022a, tablet charger 1022b, and/or other charging cables with different types of plugs. The base 1010 may also include an alignment mechanism surrounding the opening in the base 1010 that locks the electrical housing 1092 in position for an external plug to be connected.

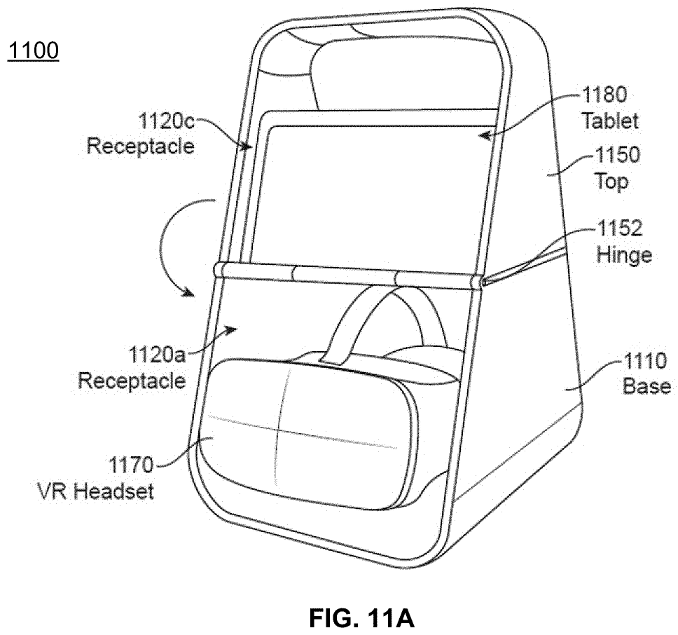

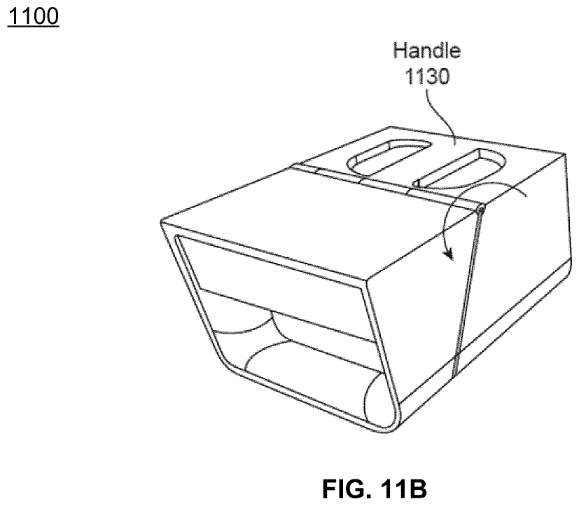

FIG. 11A illustrates an isometric view of a VR kit 1100 with a vertically oriented receptacle, according to one or more embodiments. The VR kit 1100 includes a base 1110 which includes a receptacle 1120a. The receptacle 1120 is a hollowed cavity in the base 1110 in which a VR headset, for example the VR headset 1170, may be placed for storage or transportation. Additionally, the VR kit 1100 includes a receptacle 1120c configured to store a display, for example a tablet 1180. The receptacle 1120c may be a sleeve with a single opening within which the tablet 1180 may be inserted and completely covered. Alternatively, the receptacle 1120c may be a frame which supports the tablet 1180 while leaving the screen of the tablet 1180 exposed. Regardless of the particular design, functionally, the receptacle 1120c is secures the table 1180 in a horizontal or vertical orientation during the storage or transportation of the VR kit 1100.

The receptacle 1120c is coupled to the base 1110 by a hinge 1152 which enables the receptacle 1120c to rotate about a horizontal axis. Rotation about a horizontal axis by the hinge 1152 causes the receptacle 1120c to oscillate between a closed configuration (not shown) and an open configuration (illustrated in FIG. 11A). In the closed configuration, the edges of the receptacle 1120c coupled to the edges of the base 1110 to hold the VR headset 1170 and the tablet 1180 in a fixed position within the base 1110. In the open configuration, a user may remove the VR headset 1170 from the receptacle 1120a for use. Additionally, in the open configuration, the receptacle 1120c is configured to lock into a display position on the top 1150 of the VR kit 1100. In some embodiments, the locking mechanism used to hold the receptacle 1120c in the display position is a component of the hinge, whereas in other embodiments the locking mechanism may be a component of the top 1150. Once mounted in the display position, the receptacle 1120c and the tablet 1180 are positioned such that a user operating the VR headset may use the tablet as a display panel.

FIG. 11B illustrates an isometric view of a VR kit 1100 with an alternate handle 1130, according to one or more embodiments. Consistent with the description of the VR kit 1100 illustrated in FIG. 11A, FIG. 11B illustrates a vertically oriented VR kit with a top and a base and a receptacle that rotates about a hinge. Whereas the handle illustrated in FIG. 11A is a distinct component positioned above the top 1110 of the VR kit 1100, the handle 1130 illustrated in FIG. 11B is a gripped design integrated into a face of the top of the kit. In addition to the design of the handle 1130, the handle may be designed in any other fashion that accommodates or improves a user's ability to hold or transport the kit 1100.

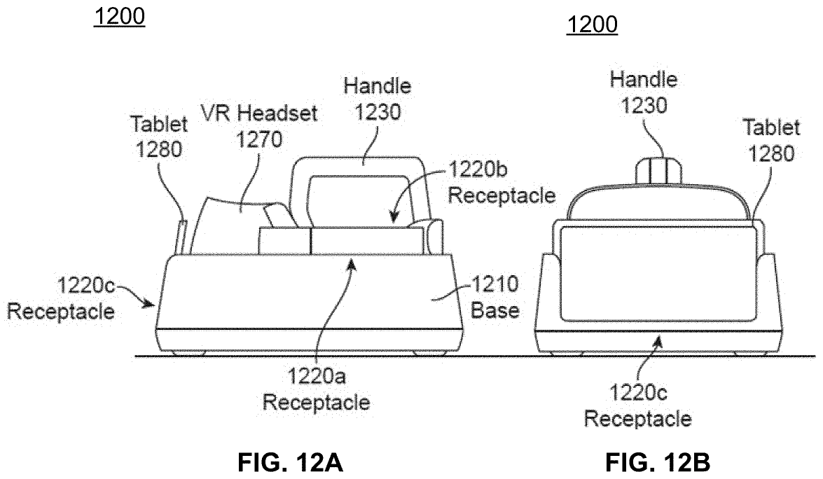

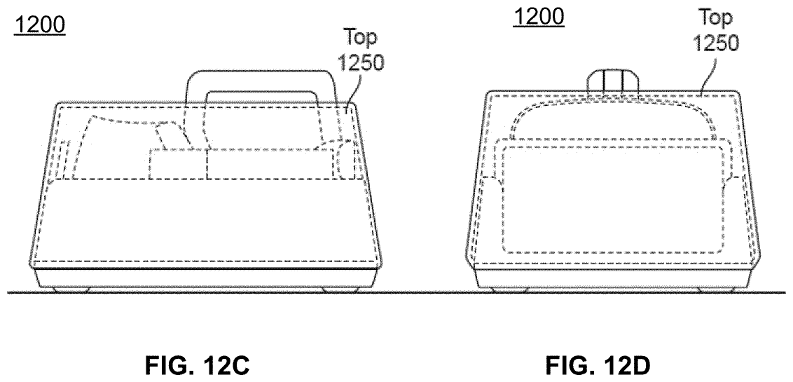

FIG. 12A illustrates a side view of a VR kit 1200 with a handle extending upward, according to one or more embodiments. The VR kit 1200 includes a base 1210 with three receptacles 1220a, 1220b, and 1220c. The VR headset 1270 is stored in one receptacle that is configured such that the VR headset 1270 is placed face down relative in the base 1210. The receptacle 1220c is designed to securely store a tablet 1280. The handle 1230 is designed to protrude in an upward direction relative to the base 1210. The handle 1230 may be coupled to the base 1210 between one edge of the base 1210 and the VR headset 1270 such that the handle does not obstruct access to the VR headset 1270. FIG. 12B illustrates a front view of a VR kit 1200, according to one or more embodiments.

FIG. 12C illustrates a side view of a VR kit 1200 with a removable top 1250, according to one or more embodiments. The top 1250 may be comprised of the same, substantially similar, or different material as the base 1210. The top 1250 may also be designed to be transparent to enable observation of components stored within VR kit 1200. The top 1250 may additionally be coupled to the base 1210 using one or more attachment mechanisms. FIG. 12D illustrates a front view of a VR kit 1200 with a removable top 1250, according to one or more embodiments.

### 7. Additional Considerations

The foregoing description of the embodiments has been presented for the purpose of illustration; it is not intended to be exhaustive or to limit the patent rights to the precise forms disclosed. Persons skilled in the relevant art can appreciate that many modifications and variations are possible in light of the above disclosure.

Any of the steps, operations, or processes described herein may be performed or implemented with one or more hardware or software modules, alone or in combination with other devices. In one embodiment, a software module is implemented with a computer program product comprising a computer-readable medium containing computer program code, which can be executed by a computer processor for performing any or all of the steps, operations, or processes described.

Embodiments may also relate to an apparatus for performing the operations herein. This apparatus may be specially constructed for the required purposes. Such a computer program may be stored in a non-transitory, tangible computer readable storage medium, or any type of media suitable for storing electronic instructions, which may be coupled to a computer system bus. Furthermore, any computing systems referred to in the specification may include a single processor or may be architectures employing multiple processor designs for increased computing capability.

Finally, the language used in the specification has been principally selected for readability and instructional purposes, and it may not have been selected to delineate or circumscribe the patent rights. It is therefore intended that the scope of the patent rights be limited not by this detailed description, but rather by any claims that issue on an application based hereon. Accordingly, the disclosure of the embodiments is intended to be illustrative, but not limiting, of the scope of the patent rights, which is set forth in the following claims.

What is claimed is:

1. A device for storing a virtual reality system comprising:
a base including a set of receptacles configured to retain a set of components of a virtual reality platform, the set of receptacles recessed within at least one of a set of surfaces of the base; and
a handle coupled to the base and operable between a set of modes, wherein in at least one of the set of modes, a contact position of the handle is aligned with a center of gravity of the device and contents of the device.

2. The device of claim 1, wherein the set of receptacles comprise:
a first receptacle recessed into a front face of the base to secure an electronic device; and
at least one receptacle recessed into an upper broad surface of the base to secure a headset.

3. The device of claim 1, wherein the handle further comprises an adjustment mechanism that translates the handle to maintain alignment with the center of gravity when components are removed from one or more receptacles.

4. The device of claim 1, wherein the base further comprises an electrical subsystem that supplies power to each receptacle to charge a component retained within the receptacle.

5. The device of claim 1, further comprising:
a cage removably coupled to the base to protect components retained in the receptacles of the base; and
a sanitation system integrated into an interior face of the cage, wherein the sanitation system applies UV light to components retained in each receptacle.

6. The device of claim 1, further comprising:
a shield that protects components retained in receptacles of the base from extensive exposure to light; and
a shield control mechanism that transitions the shield to a position to block an component of the VR system from light in response to a change in position of the handle.

7. The device of claim 1, wherein the set of receptacles are designed in a foam layer inserted into a cavity of the base.

8. The device of claim 1, further comprising:
a top that couples to the base to secure components in position within each receptacle;
and a lock configured to secure the top in a coupled position relative to the base.

9. The device of claim 1, wherein each receptacle comprises an electric charger configured to charge a component retained in the receptacle.

10. The device of claim 1, wherein the base is oriented vertically with a first receptacle configured to retain a headset and a second receptacle coupled to the base by a hinge, wherein rotation of the second receptacle about the hinge locks the second receptacle into a display position above the base.

11. The device of claim 1, wherein the handle is integrated into a lid coupled to the base by a hinge.

12. A device for storing a virtual reality system comprising:
a base including a set of receptacles configured to retain a set of components of a virtual reality platform, the set of receptacles recessed within at least one of a set of surfaces of the base, wherein the set of receptacles comprise a first receptacle recessed into a front face of the base to secure an electronic device and at least one receptacle recessed into an upper broad surface of the base to secure a headset; and
a handle coupled to the base and operable between a set of modes, wherein in at least one of the set of modes, a contact position of the handle is aligned with a center of gravity of the device and contents of the device.

13. The device of claim 12, wherein the handle further comprises an adjustment mechanism that translates the handle to maintain alignment with the center of gravity when components are removed from one or more receptacles.

14. The device of claim 12, wherein the base further comprises an electrical subsystem that supplies power to each receptacle to charge a component retained within the receptacle.

15. The device of claim 12, further comprising:
a cage removably coupled to the base to protect components retained in the receptacles of the base; and
a sanitation system integrated into an interior face of the cage, wherein the sanitation system applies UV light to components retained in each receptacle.

16. The device of claim 12, further comprising:
a shield that protects components retained in receptacles of the base from extensive exposure to light; and
a shield control mechanism that transitions the shield to a position to block an component of the VR system from light in response to a change in position of the handle.

17. The device of claim 12, wherein the set of receptacles are designed in a foam layer inserted into a cavity of the base.

18. The device of claim 12, further comprising:
a top that couples to the base to secure components in position within each receptacle;
and a lock configured to secure the top in a coupled position relative to the base.

19. The device of claim 12, wherein the base is oriented vertically with a first receptacle configured to retain a headset and a second receptacle coupled to the base by a hinge, wherein rotation of the second receptacle about the hinge locks the second receptacle into a display position above the base.

20. A device for storing a virtual reality system comprising:
a base including a set of receptacles configured to retain a set of components of a virtual reality platform, the set of receptacles recessed within at least one of a set of surfaces of the base, wherein the base is oriented vertically with a first receptacle of the set configured to retain a headset and a second receptacle of the set coupled to the base by a hinge such that rotation of the second receptacle about the hinge locks the second receptacle into a display position above the base; and
a handle coupled to the base and operable between a set of modes, wherein in at least one of the set of modes, a contact position of the handle is aligned with a center of gravity of the device and contents of the device.
